SEQUENCE LISTING (14 sequences)

SEQ ID NO: 1 - protein, 91 aa, Bacteriophage X174
MVRWTLWDTLAFLLLLSLLLPSLLIMFIPSTFKRPVSSWKALNLRKTLLMASSVRLKPLNCSRLPCVYAQETLTFLLTQKKTCVKNYVQKE

SEQ ID NO: 2 - DNA, 81 bp, Escherichia coli
  source 1..81: mol_type = genomic DNA
ccacaaagagtcacagggattgagtgttgaaatgatccggatgagcatgtatctttatggttatgttataacataacaggt

SEQ ID NO: 3 - DNA, 12 bp, Synthetic construct
  source 1..12: mol_type = other DNA
aaagaggagaaa

SEQ ID NO: 4 - DNA, 93 bp, Synthetic construct
  source 1..93: mol_type = other DNA
ccacaaagagtcacagggattgagtgttgaaatgatccggatgagcatgtatctttatggttatgttataacataacaggtaaagaggagaaa

SEQ ID NO: 5 - DNA, 364 bp, Escherichia coli
  source 1..364: mol_type = genomic DNA
taacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatg

SEQ ID NO: 6 - DNA, 376 bp, Synthetic construct
  source 1..376: mol_type = other DNA
taacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatgaaagaggagaaa

SEQ ID NO: 7 - protein, 153 aa, Homo sapiens [sequence omitted]

SEQ ID NO: 8 - protein, 134 aa, Homo sapiens
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 9 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 10 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 11 - protein, 198 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTR

SEQ ID NO: 12 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 13 - protein, 218 aa, Homo sapiens
MSFVGENSGVKMGSEDWEKDEPQCCLEDPAGSPLEPGPSLPTMNFVHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 14 - DNA, 81 bp, Escherichia coli
  source 1..81: mol_type = genomic DNA
ccacaaatagtcacagtgattgggtgttgaaatgatccggatgagcatgtatctttacggttatgttataacataacaggt

(19) United States
(12) Patent Application Publication
(10) Pub. No.: US 2025/0339476 A1
(43) Pub. Date: Nov. 6, 2025

(54) ENGINEERED BACTERIAL THERAPEUTICS FOR THE TREATMENT OF GASTROINTESTINAL DISEASE

(71) Applicant: Northwestern University, Evanston, IL (US)

(72) Inventors: Arthur Benjamin Prindle, Evanston, IL (US); Johnathan Xia, Evanston, IL (US)

(21) Appl. No.: 19/198,291

(22) Filed: May 5, 2025

Related U.S. Application Data
(60) Provisional application No. 63/642,227, filed on May 3, 2024.

(51) Int. Cl. A61K 35/741; A61K 38/20; A61P 1/04; C07K 16/24

(52) U.S. Cl. CPC A61K 35/741; A61K 38/2026; A61K 38/2033; A61K 38/204; A61K 38/2066; A61K 38/2073; A61K 38/2086; A61P 1/04; C07K 16/241; C07K 16/244; C07K 2319/02

(57) ABSTRACT

Provided herein are engineered bacterial therapeutics and methods of use thereof for treating gastrointestinal disease.

### PRIORITY STATEMENT

This application claims priority to U.S. Provisional Patent Application No. 63/642,227, filed May 3, 2024, the entire contents of which are incorporated herein by reference for all purposes.

### SEQUENCE LISTING

The text of the computer readable sequence listing filed herewith, titled “NWEST_43073_202_Corrected_SequenceListing.xml”, created Jun. 8, 2025, having a file size of 15,440 bytes, is hereby incorporated by reference in its entirety.

### FIELD

Provided herein are engineered probiotics that selectively release therapeutic antibodies in the presence of calprotectin, and methods of use thereof for treatment of inflammatory disease.

### BACKGROUND

Inflammatory bowel disease (IBD) is a family of incurable autoimmune diseases that involve chronic inflammatory flares of the GI tract and can lead to colorectal cancer when left untreated. The current cornerstone of IBD treatment is the use of antibodies designed to block pr-inflammatory cytokines implicated in IBD. However, most of these therapeutics are delivered systemically, resulting in serious side effects due to immunosuppression. Thus, there is a major need to effective treatment options for IBD with minimal side effects.

### SUMMARY

Herein, E. coli was engineered to produce and selectively release therapeutic antibodies in the GI tract only in the presence of calprotectin, the clinical gold standard biomarker of IBD. These engineered probiotics have the potential to become living medicines for long-term disease treatment in patients with gastrointestinal disease, such as IBD.

In some aspects, provided herein is an engineered bacterial therapeutic comprising a bacterial host cell expressing a protein for treatment of gastrointestinal disease, and a lysis element operably linked to a calprotectin-responsive promoter. The lysis element induces lysis of the bacterial host cell and release of the protein for treatment of gastrointestinal disease in the presence of calprotectin.

In some embodiments, the protein for treatment of gastrointestinal disease is an anti-inflammatory cytokine or an antibody. In some embodiments, the anti-inflammatory cytokine is interleukin-4 (IL-4), interleukin-5 (IL-5), interleukin-6 (IL-6), interleukin-10 (IL-10), interleukin-11 (IL-11), interleukin-13 (IL-13), interleukin-35 (IL-35), or interleukin-37 (IL-37). For example, in some embodiments the anti-inflammatory cytokine comprises a sequence having at least 90% identity to SEQ ID NO: 10. In some embodiments, the antibody is an anti-TNF-α antibody, an anti-IL-12 antibody, or an anti-IL-23 antibody. In some embodiments, antibody comprises the anti-TNF-α TNF antibody infliximab, adalimumab, golimumab, certolizumab, or ozoralizumab. In some embodiments, the antibody comprises the anti-IL-12 antibody ustekinumab. In some embodiments, the antibody comprises the anti-IL-23 antibody guselkumab, brazikumab, mirikizumab, risankizumab, or tildrakizumab.

In some embodiments, the lysis element is a phage-derived lysis element. For example, in some embodiments the phase-derived lysis element comprises an amino acid sequence having at least 90% identity with SEQ ID NO: 1.

In some embodiments, the bacterial host cell comprises a probiotic bacterial strain. In some embodiments, the bacterial host cell comprises E. coli. For example, in some embodiments the bacterial host cell comprises E. coli Nissle 1917 (EcN).

In some embodiments, the calprotectin-responsive promoter comprises ykgMO.

In some embodiments, the antibody for the treatment of gastrointestinal disease is conjugated to a signal peptide.

In some embodiments, the gastrointestinal disease comprises inflammatory bowel disease, a gastrointestinal infection, or Celiac disease.

In some aspects, provided herein is a pharmaceutical composition comprising an engineered bacterial therapeutic described herein.

The engineered bacterial therapeutics and pharmaceutical compositions provided herein find use in methods of treating gastrointestinal disease in a subject. In some embodiments, provided herein is a method of treating a gastrointestinal disease in a subject, comprising providing to the subject a pharmaceutical composition herein. In some embodiments, the gastrointestinal disease comprises inflammatory bowel disease, a gastrointestinal infection, or Celiac disease. In some embodiments, the subject is a mammal. In some embodiments, the subject is a human.

### BRIEF DESCRIPTION OF THE DRAWINGS

FIG. 1A is a schematic showing biological occurrences during IBD flares, which result in increased calprotectin and TNF alpha at the site of inflammation. FIG. 1B is a schematic showing an exemplary engineered E. coli provided herein, referred to as CS-EcN. CS-EcN uses a calprotectin-responsive promoter to deliver therapeutic proteins, such as anti-TNF-alpha antibodies, in the GI tract in response to calprotectin, and thus selectively deliver therapeutics during an IBD flare.

FIG. 2 shows calprotectin-induced lysis using the engineered E. coli strain EcN herein containing a calprotectin-responsive promoter (ykgMO) upstream of a phage-derived lysis element, specifically the E protein from the bacteriophage ΦX174. This lysis element is shown in the schematic as ΦX174E.

FIG. 3 is a schematic representation of how calprotectin-induced lysis is prevented in the presence of zinc ions.

FIG. 4 shows effective prevention of calprotectin-induced lysis in the presence of a sufficient concentration of zinc ions.

FIG. 5A shows a representative schematic of suitable construct components for the engineered bacteria described herein. The therapeutic agent was placed downstream of an IPTG-sensitive tac promoter (Ptac) that controls production of the therapeutic agent. An ompA signal peptide was placed downstream of the tac promoter and upstream of the therapeutic agent to target to the periplasm for proper folding of the therapeutic. A histidine tag was placed downstream of the therapeutic to facilitate purification for evaluation of protein levels. FIG. 5B shows a western blot verifying production of the therapeutic agent certolizumab. Results are quantified in in FIG. 5C.

FIG. 6A is a schematic showing the exemplary HEK-blue TNF-alpha functional assay used herein. Functionality results are presented in FIG. 6B and a comparison of functionality to pure certolizumab is shown in FIG. 6C.

FIG. 7A shows bacterial production of the therapeutic agent ozoralizumab (anti-TNF-alpha nanobody). FIG. 7B shows functionality results from the HEK-blue TNF-alpha functional assay.

FIG. 8 shows successful production of certolizumab pegol (Cimzia®) in engineered bacteria, validated by western blot. Data shows purification of BL21 WT, an engineered calprotectin-responsive bacteria described herein (pJBL5034-lac-cimzia) in induced (calprotectin) and uninduced (no calprotectin) conditions. The first two elution fractions of each were evaluated and the concentration equalized to 200 micrograms per milliliter using 12/8 BCA. The expected size of certolizumab pegol is 25 kDa.

FIGS. 9A-9B show successful production of certolizumab pegol in engineered bacteria, validated by ELISA. FIG. 9A shows raw numbers from the ELISA and FIG. 9B shows data multiplied by the dilution factor.

FIG. 10 shows successful production of IL-10 (e.g. IL-10 release) in engineered bacteria, evaluated by ELISA. Data shows purification of BL21 WT and pSC02-IL-10-ompA de la Rosa induced and uninduced. Dilution series of total concentrations of 100 ng/mL, 10 ng/mL, and 1 ng/mL were ran.

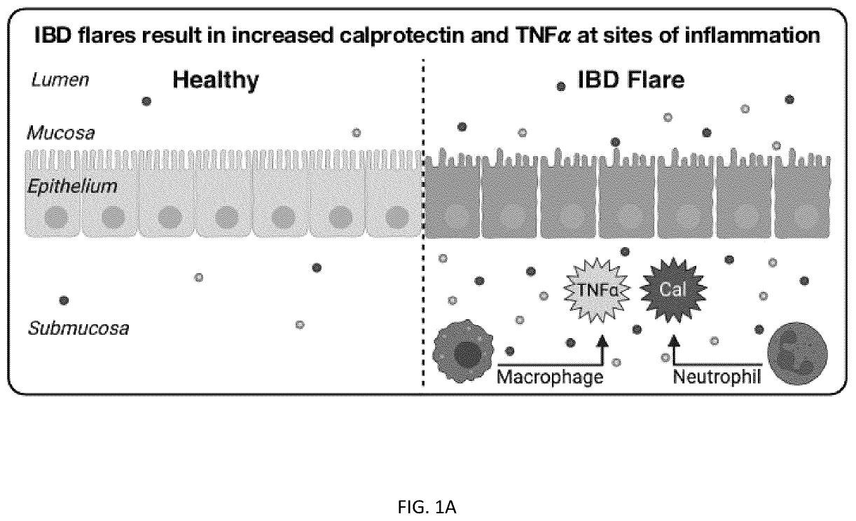

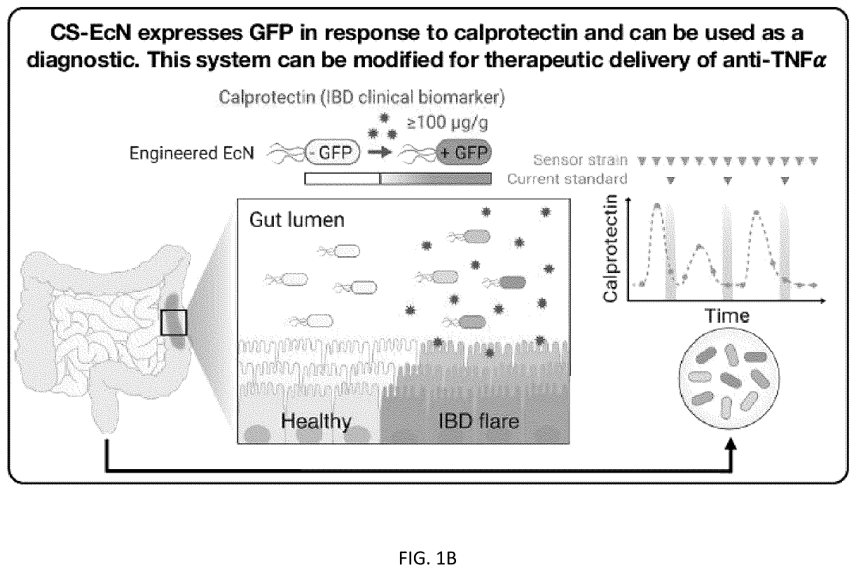

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “antibody” refers to a whole antibody molecule or a fragment thereof (e.g., fragments such as scFv, Fab, Fab′, and F(ab′)₂). An antibody may be a polyclonal or monoclonal antibody, a chimeric antibody, a humanized antibody, a human antibody, etc. An “antibody fragment” (which is encompassed within the term “antibody” as used herein) refers to a portion of a full-length antibody, including at least a portion antigen binding region or a variable region. Antibody fragments include, but are not limited to, Fab, Fab′, F(ab′)₂, Fv, scFv, Fd, diabodies, nanobodies, and other antibody fragments that retain at least a portion of the variable region of an intact antibody (see, e.g., Hudson et al., Nat. Med., 9: 129-134 (2003)). For example, a “Fab” fragment comprises one light chain and the CHi and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen binding region. In certain instances, a single variable region (e.g., a heavy chain variable region or a light chain variable region) may have the ability to recognize and bind antigen. Other antibody fragments will be understood by skilled artisans.

As used herein, the term “gastrointestinal disease” is used in the broadest sense and is inclusive of any disease/disorder or infection of the gastrointestinal system. In some embodiments, the gastrointestinal disease is characterized by mucosal damage and increased calprotectin production. In some embodiments, the gastrointestinal disease is inflammatory bowel disease. In some embodiments, the gastrointestinal disease is Celiac disease. In some embodiments, the gastrointestinal disease is a gastrointestinal infection, such as Clostridium difficile.

As used herein, the term “subject” broadly refers to any animal, including but not limited to, human and non-human animals (e.g., dogs, cats, cows, horses, sheep, pigs, poultry, fish, crustaceans, etc.). In some embodiments, the subject is a mammal. In some embodiments, the subject is a human. In some embodiments, the subject has or is suspected of having inflammatory bowel disease.

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely reduces/cures/ameliorates the disease and/or adverse symptom attributable to the disease.

The term “pharmaceutical composition” as used herein refers to a composition comprising at least one pharmaceutically-active agent, chemical substance or drug. The pharmaceutical composition may be in solid or liquid form and can comprise at least one additional active agent, carrier, vehicle, excipient or auxiliary agent identifiable by the skilled person. The selection of the additional ingredients may vary depending on the intended route of administration.

The term “effective dose” or “effective amount” refers to an amount of an agent, e.g., a neutralizing antibody, that results in the reduction of symptoms in a patient, treatment of prevention of a disease or condition, or results in a desired biological outcome.

As used herein, the terms “administration” and “administering” refer to the act of giving/providing a drug, prodrug, or other agent, or therapeutic to a subject or in vivo, in vitro, or ex vivo cells, tissues, and organs.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Inflammatory Bowel Disease (IBD) is a group of autoimmune inflammatory diseases of the GI tract (including ulcerative colitis and Crohn's disease) characterized by chronic and intermittent inflammatory flares leading to symptoms such as diarrhea, abdominal pain, and rectal bleeding, which if left untreated can lead to permanent bowel damage and increased risk of colorectal cancer. Prevalence of IBD has steadily increased over time, with 6.8 million global cases of IBD being reported in 2017. The symptoms of IBD stem from an overactivation of the immune system within the gut and increased secretion of proinflammatory cytokines, especially Tumor Necrosis Factor-α (TNFα). Anti-TNFα therapeutics have been developed that successfully reduced gastrointestinal (GI) tract inflammatory response and promoted healing of gut epithelial cells. However, because these drugs are administered intravenously or subcutaneously, long term use of these systemic therapies has serious side effects of systemic immunosuppression including lymphoma and increased risk of opportunistic infections. Additionally, 40-50% of IBD patients will not respond to current systemic anti-TNFα delivery strategies, creating a gap in treatment that could potentially be addressed by local delivery in the GI tract. As such, improved methods of treating IBD are needed.

In some embodiments, provided herein is an engineered probiotic strain of Escherichia coli capable of safely colonizing the human GI tract and selectively releasing therapeutic proteins such as anti-TNFα therapeutic antibodies or IL-10, in the presence of calprotectin, an IBD biomarker. This targeted therapeutic may reduce the negative effects of systemic delivery. Moreover, the engineered bacterial therapeutic provided herein uses gene regulation to conditionally release a therapeutic only when a disease biomarker is detected, a feature that is particularly useful for IBD due to the intermittent nature of inflammatory flares. As such, the therapeutic both specifically targets the GI tract and selectively releases the therapeutic when inflammation is present.

In some aspects, provided herein is an engineered bacterial therapeutic for treatment of gastrointestinal disease. In some embodiments, provided herein is an engineered bacterial therapeutic comprising a bacterial host cell expressing:
a) a protein for treatment of gastrointestinal disease, and
b) a lysis element operably linked to a calprotectin-responsive promoter.

In some embodiments, the bacterial host cell expresses a vector (e.g. plasmid) encoding the protein for treatment of gastrointestinal disease (e.g. the “therapeutic protein”), the lysis element, and the calprotectin-responsive promoter. For example, in some embodiments the bacterial host cell expresses a vector comprising a sequence encoding the protein for treatment of gastrointestinal disease, a sequence encoding the lysis element, and a sequence encoding the calprotectin-responsive promoter, wherein expression of the lysis element is operably linked to the calprotectin-responsive promoter.

In some embodiments, the lysis element is expressed and thereby induces lysis of the bacterial host cell and release of the protein for treatment of gastrointestinal disease in the presence of calprotectin. Inducing lysis in the “presence of calprotectin” indicates that a level or amount of calprotectin is sufficient to induce said lysis, e.g. a level or amount of calprotectin is equal to or above a threshold or cutoff value. For example, a comparatively low level of calprotectin (e.g. a basal level of calprotectin, not affiliated with any particular disease state) may be insufficient to induce lysis of the bacterial host cell, whereas a level of calprotectin equal to or above a threshold value (e.g. an elevation in calprotectin levels, for example an elevated level associated with a disease state) is sufficient to induce lysis. Accordingly, the engineered bacteria provided herein selectively release the protein for treatment of gastrointestinal disease (also referred to as the “therapeutic protein”) only in the presence of a sufficient level or amount of calprotectin, which is elevated, for example, during IBD flares. This minimizes unnecessary exposure to the therapeutic protein unless a flare is actively occurring (or other disease conditions are present such that calprotectin is elevated in the subject). This avoids unwanted side effects and results in selective, purposeful administration directly to the GI tract during an inflammatory state. This is advantageous in that systemic administration, which is often associated with negative side effects, is avoided.

In some embodiments, the lysis element is a phage-derived lysis element. Phage-derived lysis elements are proteins produced by bacteriophages (i.e. phages) to lyse their host bacterial cells, thus releasing new phage cycles and promoting the phage replication cycle. Any suitable phage-derived lysis element may be used. In some embodiments, the phage-derived lysis element is derived from phi X 174, or ΦX174, a single-stranded DNA virus that infects E. coli. In some embodiments, the phage-derived lysis element is the lysis protein E from ΦX174, referred to herein as ΦX174E. The sequence of ΦX174E is as follows:
MVRWTLWDTLAFLLLLSLLLPSLLIMFIPSTFKRPVSSWKALNLRKTLLMASSVR LKPLNCSRLPCVYAQETLTFLLTQKKTCVKNYVQKE (SEQ ID NO: 1). In some embodiments, the phage derived lysis element comprises a sequence having at least 80% identity to SEQ ID NO: 1. For example, in some embodiments the phage-derived lysis element comprises a sequence having at least 80%, 85%, 90%, 91%, 92%, 93%, 94%, 95%, 96%, 97%, 98%, 99%, or 100% identity with SEQ ID NO: 1.

The lysis element is operably linked to a calprotectin-responsive promoter. The calprotectin-responsive (also referred to herein as “calprotectin-sensitive”) promoter may be a promoter of any calprotectin-responsive gene (e.g. calprotectin-sensitive) gene. A calprotectin-responsive or a calprotectin-sensitive gene are used interchangeably herein and refer to a gene that is differentially expressed in conditions with and without calprotectin or differentially expressed in conditions that have achieved or not achieved a threshold level of calprotectin. In some embodiments, a calprotectin-responsive gene is upregulated in response to a sufficient level or amount of calprotectin in contrast to an insufficient level or amount of calprotectin. In some embodiments, the calprotectin-responsive promoter is a promoter from a calprotectin-responsive gene that is naturally (i.e. natively) expressed in the bacterial host cell. For example, in some embodiments the host cell is E. coli and the calprotectin-responsive promoter is a promoter from a calprotectin-responsive gene that is naturally (i.e. natively) expressed in E. coli. In some embodiments, the calprotectin-responsive promoter is ykgMO. For example, in some embodiments the bacterial host cell is E. coli Nissle 1917 (EcN) and the calprotectin-responsive promoter is a promoter from a calprotectin-responsive gene native to E. coli Nissle 1917 (EcN). For example, in some embodiments the bacterial host cell is E. coli (e.g. E. coli Nissle 1917 (EcN)) and the calprotectin-responsive promoter is ykgMO.

In some embodiments, the ykgMO promoter is encoded by a sequence comprising CCACAAAGAGTCACAGGGATTGAGTGTTGAAATGATCCGGATGAGCATGTATCTTTA TGGTTATGTTATAACATAACAGGT (SEQ ID NO: 2). In some embodiments, the ykgMO promoter is encoded by a sequence comprising a series of nucleotides having at least 80% (at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) sequence identity with SEQ ID NO: 2. For example, in some embodiments, the ykgMO promoter is encoded by a sequence comprising CCACAAATAGTCACAGTGATTGGGTGTTGAAATGATCCGGATGAGCATGTATCTTTA CGGTTATGTTATAACATAACAGGT (SEQ ID NO: 14).

In some embodiments, the calprotectin-responsive promoter additionally contains a ribosomal binding site. In some embodiments, the RBS is encoded by the sequence AAAGAGGAGAAA (SEQ ID NO: 3). For example, in some embodiments the calprotectin-responsive promoter is encoded by a sequence comprising CCACAAAGAGTCACAGGGATTGAGTGTTGAAATGATCCGGATGAGCATGTATCTTTA TGGTTATGTTATAACATAACAGGTAAAGAGGAGAAA (SEQ ID NO: 4). In some embodiments, the promoter is encoded by a sequence comprising a series of nucleotides having at least 80% sequence identity (at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) identity with SEQ ID NO: 4.

In some embodiments, calprotectin-responsive promoter additionally contains one or more regions of the gene from which the promoter was obtained. For example, in some embodiments the calprotectin-responsive promoter additionally contains intergenic regions upstream and/or downstream of the gene from which the promoter was obtained. In some embodiments, the promoter comprises ykgMO with intergenic regions (ykgMO-IGS) and is encoded by a sequence comprising TAACGGCAATAAACTGTTCACTTCAGTGATATTTAAAATATGCATCCTCTCCCTTTTT TGTAAGTAATTATTATATCCGTGGGAGAGGAATACACATTGTCAGGTAATCAATCAT GCTGCAATAAATCATCGGCCAGTAAAGTGGAGATAGCCTCCATTCTCGAAAAATCC ATACTCTCAGCGAAACCATCATCAATCACTCATCCAGGCGTTTATGGGAGCGTCGCC AATGGCTGCTAACAATGCCAGACTTCCCCGTTGCGGAAATTCCACATCCCACAAATA GTCACAGTGATTGGGTGTTGAAATGATCCGGATGAGCATGTATCTTTACGGTTATGT TATAACATAACAGGTAAAAATG (SEQ ID NO: 5). In some embodiments, the promoter is encoded by a sequence comprising a series of nucleotides having at least 80% (at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% m or 100%) sequence identity with SEQ ID NO: 5. In some embodiments, the promoter comprises intergenic regions and a ribosomal binding site (e.g. SEQ ID NO: 3). For example in some embodiments the promoter is encoded by a sequence comprising TAACGGCAATAAACTGTTCACTTCAGTGATATTTAAAATATGCATCCTCTCCCTTTTT TGTAAGTAATTATTATATCCGTGGGAGAGGAATACACATTGTCAGGTAATCAATCAT GCTGCAATAAATCATCGGCCAGTAAAGTGGAGATAGCCTCCATTCTCGAAAAATCC ATACTCTCAGCGAAACCATCATCAATCACTCATCCAGGCGTTTATGGGAGCGTCGCC AATGGCTGCTAACAATGCCAGACTTCCCCGTTGCGGAAATTCCACATCCCACAAATA GTCACAGTGATTGGGTGTTGAAATGATCCGGATGAGCATGTATCTTTACGGTTATGT TATAACATAACAGGTAAAAATGAAAGAGGAGAAA (SEQ ID NO: 6). In some embodiments, the promoter is encoded by a sequence comprising a series of nucleotides having at least 80% (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% m or 100%) sequence identity with SEQ ID NO: 6.

In some embodiments, the bacterial host cell is engineered such that the protein for treatment of gastrointestinal disease (e.g. the therapeutic protein) is operatively linked to a promoter. For example, in some embodiments the bacterial host cell expresses a vector comprising a sequence encoding the therapeutic protein and a sequence encoding a promoter operably linked to the therapeutic protein. In some embodiments, the promoter is native to the bacterial host cell, e.g. a promoter native to E. coli. Any suitable promoter may be used, including constitutive promoters or inducible promoters. For example, the promoter may be sensitive to IPTG, such as the tac promoter used in the accompanying examples. In some embodiments, the bacterial host cell is engineered such that the protein for treatment of gastrointestinal disease is constitutively expressed. Accordingly, in some embodiments production of the protein for treatment of gastrointestinal disease in the bacterial host cells is continuous, whereas the lysis element is only expressed under conditions with sufficient levels of calprotectin and therefore the protein is not effectively released until the appropriate environmental conditions (e.g. levels of calprotectin) is achieved.

In some embodiments, the protein for treatment of gastrointestinal disease is an anti-inflammatory cytokine or an antibody. Cytokines are a broad category of relatively small proteins that act as signaling molecules within the body, influencing the immune system and inflammation. Anti-inflammatory cytokines refer to a subset of cytokines that help to suppress/resolve inflammation. In some embodiments, the anti-inflammatory cytokine is interleukin-4 (IL-4), interleukin-5 (IL-5), interleukin-6 (IL-6), interleukin-10 (IL-10), interleukin-11 (IL-11), interleukin-13 (IL-13), interleukin-35 (IL-35), or interleukin-37 (IL-37). In some embodiments, the anti-inflammatory cytokine is a human cytokine. For example, in some embodiments the anti-inflammatory cytokine is human IL-4, human IL-5, human IL-6, human IL-10, human IL-11, human IL-13, human IL-35, or human IL-37.

In some embodiments, the anti-inflammatory cytokine is human IL-4. The sequence of human IL-4 is as follows:

(SEQ ID NO: 7)
MGLTSQLLPPLFFLLACAGNFVHGHKCDITLQEIIKTLNSLTEQKTLCT
ELTVTDIFAASKNTTEKETFCRAATVLRQFYSHHEKDTRCLGATAQQFH
RHKQLIRFLKRLDRNLWGLAGLNSCPVKEANQSTLENFLERLKTIMREK
YSKCSS

In some embodiments, the anti-inflammatory cytokine is human IL-5. The sequence of human IL-5 is as follows:

(SEQ ID NO: 8)
MRMLLHLSLLALGAAYVYAIPTEIPTSALVKETLALLSTHRTLLIANET
LRIPVPVHKNHQLCTEEIFQGIGTLESQTVQGGTVERLFKNLSLIKKYI
DGQKKKCGEERRRVNQFLDYLQEFLGVMNTEWIIES.

In some embodiments, the anti-inflammatory cytokine is human IL-6. The sequence of human IL-6 is as follows:

(SEQ ID NO: 9)
MNSFSTSAFGPVAFSLGLLLVLPAAFPAPVPPGEDSKDVAAPHRQPLTS
SERIDKQIRYILDGISALRKETCNKSNMCESSKEALAENNLNLPKMAEK
DGCFQSGFNEETCLVKIITGLLEFEVYLEYLQNRFESSEEQARAVQMST
KVLIQFLQKKAKNLDAITTPDPTTNASLLTKLQAQNQWLQDMTTHLILR
SFKEFLQSSLRALRQM.

In some embodiments, the anti-inflammatory cytokine is human IL-10. The sequence of human IL-10 is as follows:

(SEQ ID NO: 10)
MHSSALLCCLVLLTGVRASPGQGTQSENSCTHFPGNLPNMLRDLRDAFS
RVKTFFQMKDQLDNLLLKESLLEDFKGYLGCQALSEMIQFYLEEVMPQA
ENQDPDIKAHVNSLGENLKTLRLRLRRCHRFLPCENKSKAVEQVKNAFN
KLQEKGIYKAMSEFDIFINYIEAYMTMKIRN.

In some embodiments, the anti-inflammatory cytokine is human IL-11. The sequence of human IL-11 is as follows:

(SEQ ID NO: 11)
MNCVCRLVLVVLSLWPDTAVAPGPPPGPPRVSPDPRAELDSTVLLTRSL
LADTRQLAAQLRDKFPADGDHNLDSLPTLAMSAGALGALQLPGVLTRLR
ADLLSYLRHVQWLRRAGGSSLKTLEPELGTLQARLDRLLRRLQLLMSRL
ALPQPPPDPPAPPLAPPSSAWGGIRAAHAILGGLHLTLDWAVRGLLLLK
TRL.

In some embodiments, the anti-inflammatory cytokine is human IL-13. The sequence of human IL-13 is as follows:

(SEQ ID NO: 12)
MHPLLNPLLLALGLMALLLTTVIALTCLGGFASPGPVPPSTALRELIEE
LVNITQNQKAPLCNGSMVWSINLTAGMYCAALESLINVSGCSAIEKTQR
MLSGFCPHKVSAGQFSSLHVRDTKIEVAQFVKDLLLHLKKLFREGQFN.

In some embodiments, the anti-inflammatory cytokine is human IL-35. IL-35 is a heterodimeric cytokine within the IL-12 family, formed by the IL-12A subunit (also referred to as p35, accession no. P29459) and EBI3 (accession no. NP_005746.2) subunits.

In some embodiments, the anti-inflammatory cytokine is human IL-37. The sequence of human IL-37 is as follows:

(SEQ ID NO: 13)
MSFVGENSGVKMGSEDWEKDEPQCCLEDPAGSPLEPGPSLPTMNFVHTS
PKVKNLNPKKFSIHDQDHKVLVLDSGNLIAVPDKNYIRPEIFFALASSL
SSASAEKGSPILLGVSKGEFCLYCDKDKGQSHPSLQLKKEKLMKLAAQK
ESARRPFIFYRAQVGSWNMLESAAHPGWFICTSCNCNEPVGVTDKFENR
KHIEFSFQPVCKAEMSPSEVSD.

In some embodiments, the anti-inflammatory cytokine comprises a sequence having at least 80% identity to SEQ ID NO: 7, SEQ ID NO: 8, SEQ ID NO: 9, SEQ ID NO: 10, SEQ ID NO: 11, SEQ ID NO: 12, or SEQ ID NO: 13. In some embodiments, the bacterial host cell expresses a vector containing a sequence encoding the anti-inflammatory cytokine. For example, in some embodiments the bacterial host cell expresses a vector containing a nucleotide sequence encoding an amino acid sequence for any of the above anti-inflammatory cytokines.

In some embodiments, the protein for treatment of gastrointestinal disease is an antibody (e.g. an antibody for treatment of gastrointestinal disease). The term “antibody” as used herein includes antibodies and antibody fragments. Any suitable antibody for the treatment of gastrointestinal disease can be used. Particularly useful antibodies include those targeting inflammatory pathways to reduce inflammation, including an anti-TNF-α antibodies, anti-IL-12 antibodies, anti-IL-23 antibodies, and the like.

In some embodiments, the antibody is an anti-TNF-α antibody. For example, in some embodiments the anti-TNF-α antibody comprises infliximab, adalimumab, golimumab, certolizumab (e.g. certolizumab pegol), or oralizumab.

In some embodiments, the antibody is an anti-IL-12 antibody. In some embodiments, the anti-IL-12 antibody comprises ustekinumab.

In some embodiments, the antibody is an anti-IL-23 antibody. In some embodiments, the anti-IL-23 antibody comprises guselkumab, brazikumab, mirikizumab, risankizumab, or tildrakizumab.

In some embodiments, the protein for the treatment of gastrointestinal disease is conjugated to a signal peptide. Generally speaking, the signal peptide directs the protein to the appropriate compartment of the cell, such as for proper folding and/or secretion. The exemplary signal peptide ompA is used herein, however any suitable signal peptide may be used. In some embodiments, the signal peptide is MalE, OmpA, PhoA, DsbA, or PelB. In some embodiments, the bacterial host cell expresses a vector containing a nucleotide sequence encoding the protein for treatment of gastrointestinal disease and a nucleotide sequence encoding the signal peptide which directs the protein to the desired cell compartment.

Any suitable bacterial host cell may be used. In some embodiments, the bacterial host cell comprises a probiotic bacterial strain. As used herein, the term “probiotic” refers to a bacteria that confer a health benefit to the host. In some embodiments, the bacterial host cell comprises a probiotic bacterial strain that colonizes in the human gut. In some embodiments, the bacterial host cell comprises a probiotic strain selected from the genera Lactobacillus, Bifidobacterium, Saccharomyces, Streptococcus, Enterococcus, Escherichia, and Bacillus. In some embodiments, the bacterial host cell comprises E. coli. In some embodiments, the bacterial host cell comprises a probiotic E. coli strain. In some embodiments, the bacterial host cell comprises E. coli Nissle 1917 (EcN). In some embodiments, the bacterial host cell comprises E. coli strain BL21.

The engineered bacterial biosensors provided herein find use in methods of treating gastrointestinal disease (e.g. inflammatory bowel disease, Clostridium difficile infection, Crohn's disease, etc.) in a subject. The gastrointestinal disease can be any gastrointestinal disease/disorder or infection affiliated with increased calprotectin production. In some embodiments, the gastrointestinal disease comprises inflammatory bowel disease. In some embodiments, the gastrointestinal disease comprises a gastrointestinal infection, such as Clostridium difficile. In some embodiments, the gastrointestinal disease comprises Celiac disease.

In some aspects, provided herein is a method comprising providing the engineered bacterial therapeutic of any one of the preceding claims to a subject having or suspected of having a gastrointestinal disease. In some embodiments, providing the engineered bacterial therapeutic to the subject treats the gastrointestinal disease in the subject.

The engineered bacterial therapeutic may be formulated into a suitable pharmaceutical composition for administration to the subject. In some embodiments, the engineered bacterial therapeutic is formulated for parenteral administration (e.g. by injection, including intravenous, intramuscular, subcutaneous, intraarterial, etc.). In some embodiments, the engineered bacterial therapeutic is formulated for oral administration to the subject. The pharmaceutical composition may contain any one or more suitable additional ingredients, including excipients, carriers, stabilizers, preservatives, sweeteners, and the like, depending on the mode of administration.

Any suitable dose and dosing regimen of the pharmaceutical composition may be used in the method of treating gastrointestinal disease. The dose and dosing regimen may depend on the age of the subject, the weight of the subject, the severity of symptoms, and the like. Generally speaking, the dose of the pharmaceutical composition should be sufficient such that the bacterial host cell reaches the gut and delivers an effective dose of the therapeutic protein after expression and cell lysis.

In some embodiments, the pharmaceutical composition is administered (e.g. provided) to the subject daily. In some embodiments, the pharmaceutical composition is provided to the subject more than once per day. In some embodiments, the pharmaceutical composition is provided to the subject less than once per day. For example, in some embodiments the pharmaceutical composition is provided to the subject daily, every 2 days, every 3 days, every 4 days, every 5 days, every 6 days, every 7 days, every 8 days, every 9 days, every 10 days, every 11 days, every 12 days, every 13 days, every 14 days, every 2 weeks, every 3 weeks, every 4 weeks, or less than once per month. In some embodiments, the pharmaceutical composition is provided to the subject during an episode of symptoms of the gastrointestinal disease (e.g. during an IBD flare). In some embodiments, the pharmaceutical composition is provided to the subject prophylactically, e.g. prior to or in between flare ups.

### EXPERIMENTAL

### Example 1

Inflammatory Bowel Disease (IBD) is a group of autoimmune diseases of the GI tract (including ulcerative colitis and Crohn's disease) characterized by relapsing and remittent flares of gut inflammation, leading to symptoms such as diarrhea, abdominal pain, and rectal bleeding, which if left untreated can lead to permanent bowel damage and increased risk of colorectal cancer. The symptoms of IBD stem from an overactivation of the immune system within the gut and increased secretion of proinflammatory cytokines, especially the master regulator of inflammation Tumor Necrosis Factor-α (TNFα). Therapeutics that block TNFα have been developed that successfully reduced gastrointestinal (GI) tract inflammatory response and promoted healing of gut epithelial cells. However, because these drugs are administered intravenously or subcutaneously, systemic immunosuppression leads to serious side effects including increased risk of opportunistic infections and non-Hodgkin's lymphoma. Additionally, 40-50% of IBD patients will not respond to current anti-TNFα therapeutics, creating a major gap in treatment.

IL-10 is a key regulatory cytokine with anti-inflammatory effects. IL-10R mutations in humans are associated with early onset IBD, suggesting an important role in IBD pathogenesis. Moreover, IL-10 knockouts in mice lead to spontaneous development of colitis, further evidencing the role of IL-10 in IBD. Direct delivery of IL-10 has been explored as a therapeutic with disappointing results, which are attributed to poor delivery. As such, IL-10 has yet to be effectively targeted for IBD therapeutics.

Herein, engineered E. coli capable of safely colonizing the human GI tract and selectively releasing therapeutic proteins (e.g. therapeutic anti-TNFα antibodies, therapeutic IL-10) in the presence of the IBD biomarker calprotectin was developed. Various E. coli strains may be suitable for use in the engineered bacterial therapeutics herein. Specifically, a probiotic strain of E. coli Nissle 1917 (EcN) EcN, along with the E. coli strain BL21, was engineered to produce therapeutic proteins and selectively release the therapeutic proteins (e.g. anti-TNFα antibodies, IL-10) via activity of a phage-derived lysis element operatively linked to a calprotectin-responsive promoter, such that lysis occurs only in the presence of calprotectin.

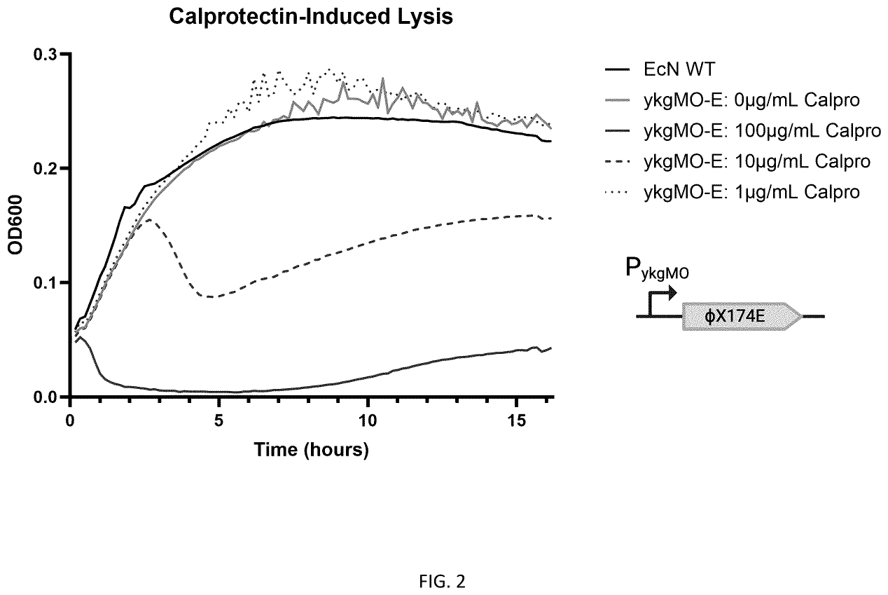

Results in FIG. 2 demonstrate effective calprotectin-induced lysis, with the clinical concentration of calprotectin defined as active inflammation of 100 μg/mL. The E. coli strain EcN was engineered to contain a calprotectin-responsive promoter (ykgMO) upstream of the phage-derived lysis element 174E, specifically the E protein from the bacteriophage ΦX174 (shown in FIG. 2 as ΦX174). Lysis at sufficient concentrations of calprotectin was confirmed, whereas lysis did not occur in EcN WT strains, engineered EcN with no calprotectin, or engineered EcN with insufficient levels of calprotectin.

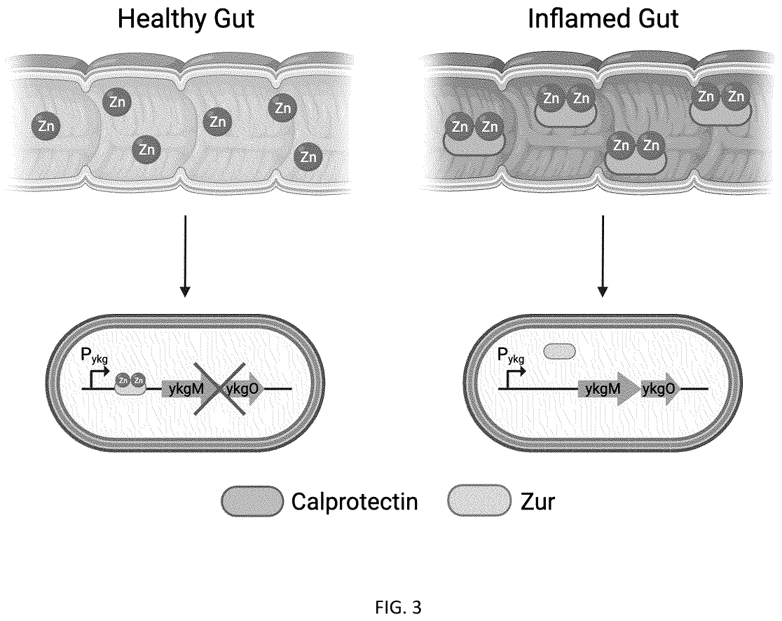

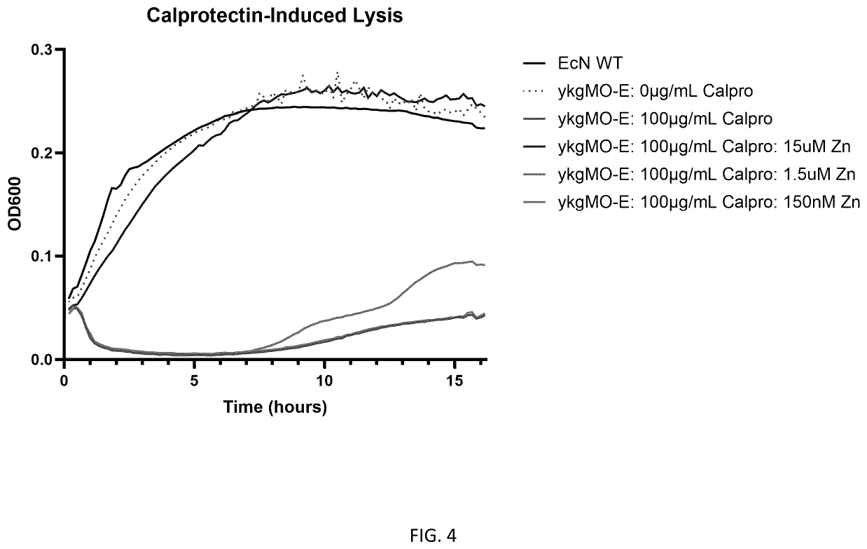

As shown in the schematic of FIG. 3, this calprotectin-induced lysis is blocked by zinc ions that are typically present in the healthy gut. These zinc ions are sequestered by calprotectin in pathological conditions such as IBD. To confirm experimentally, calprotectin-induced lysis was evaluated in the presence or absence of varying concentrations of zinc ions. The results in FIG. 4 demonstrate that lysis occurs in the presence of a sufficient concentration of calprotectin (e.g. 100 μg/mL, as established in FIG. 2) and this lysis is prevented by zinc at sufficient concentrations. Specifically, a concentration of 15 μM zinc prevented calprotectin-induced lysis, whereas 1.5 μM and 150 nM concentrations were insufficient. These results validate the use of a calprotectin-responsive ykgMO promoter upstream of a phage-derived lysis element.

After confirming calprotectin-induced lysis, E. coli was engineered to express various therapeutic proteins, including the therapeutic anti-TNFα antibody certolizumab, the therapeutic anti-TNFα antibody (e.g. nanobody) ozoralizumab, and the anti-inflammatory cytokine IL-10. The engineered bacteria herein are demonstrated to herein effectively produce the desired therapeutic agent.

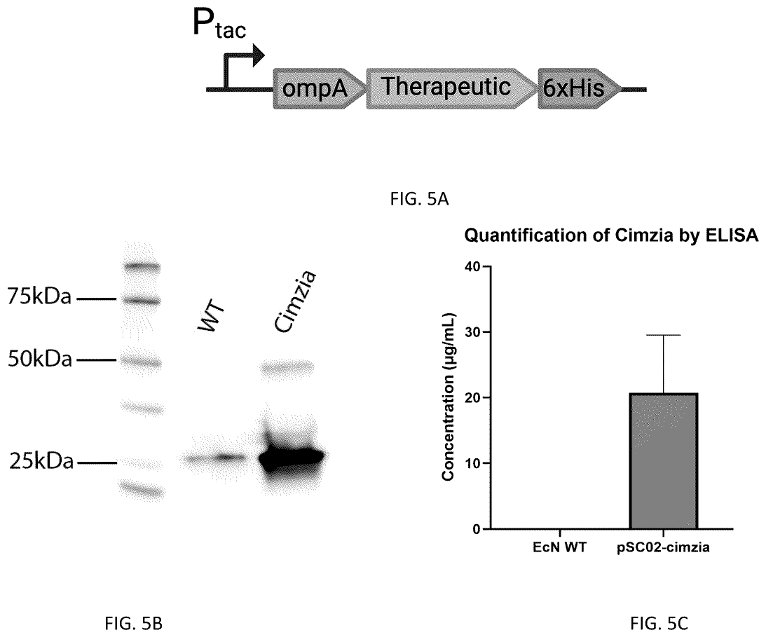

A representative schematic of suitable construct components for the engineered bacteria is shown in FIG. 5A. The therapeutic agent was placed downstream of an IPTG-sensitive tac promoter (Ptac) that controls production of the therapeutic agent. An ompA signal peptide was placed downstream of the tac promoter and upstream of the therapeutic agent to target to the periplasm for proper folding of the therapeutic. A histidine tag was placed downstream of the therapeutic to facilitate purification for evaluation of protein levels. One of more of these components (in particular the therapeutic peptide and the signal peptide) can be integrated into an engineered bacterial construct comprising a phage-derived lysis element operably liked to the calprotectin-responsive promoter (e.g. ykgMO) to create an engineered bacteria that produces a therapeutic agent and selectively lyses and releases the therapeutic agent when suitable amounts of calprotectin are present.

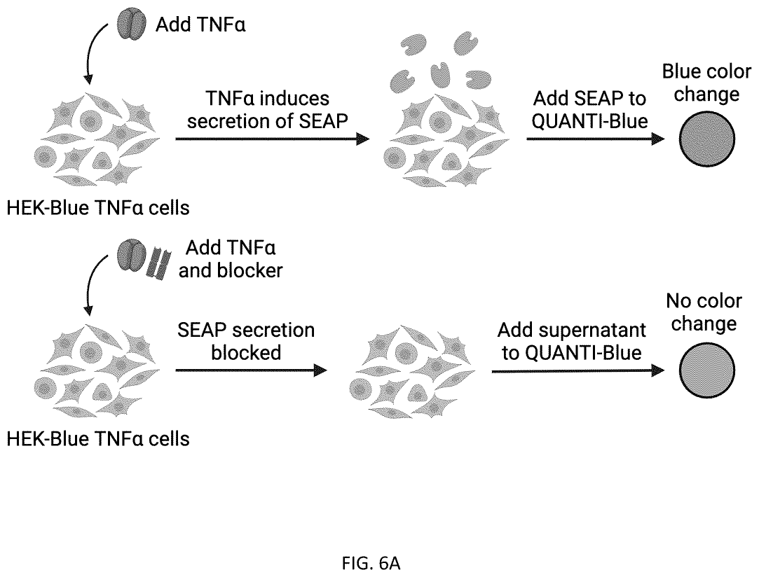

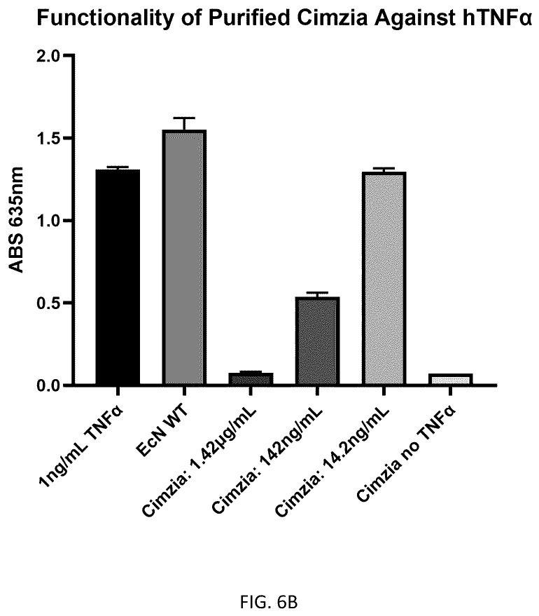

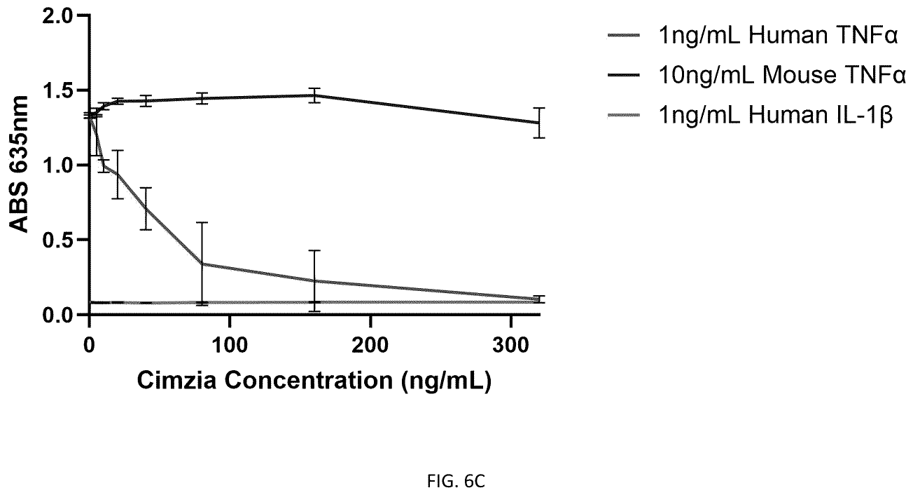

Production of the therapeutic agent certolizumab using the construct containing the exemplary components in FIG. 5A was verified by western blot and ELISA. Results are presented in FIG. 5B (raw western blot results) and quantified in FIG. 5C. These results demonstrate effective production of the exemplary therapeutic anti-TNFα antibody certolizumab. Functionality of the purified antibody was also tested. FIG. 6A is a schematic showing the exemplary HEK-blue TNF-alpha functional assay used herein. Under control conditions, TNFα induces secretion of secreted embryonic alkaline phosphatase (SEAP). Addition of the detection reagent (QUANI-blue) produces a blue color change indicative of sufficient SEAP levels. In contrast, the presence of a TNFα inhibitor (e.g. blocker) prevents/reduces/inhibits SEAP secretion, and the addition of the detection reagent fails to produce the blue color change. As such, the observance of a color change indicates that TNFα is not inhibited, whereas no color change indicates TNFα is effectively inhibited.

Functionality results using the HEK-blue functional assay are presented in FIG. 6B and a comparison of functionality to pure certolizumab is shown in FIG. 6C. As shown in FIG. 6B, in positive control conditions (e.g. the presence of TNFα alone (no certolizumab) and EcN WT cells (which do not produce certolizumab) an observable color change occurs as reflected by increased absorbance measured at 635 nm. In the negative control condition (certolizumab but no TNFα) no color change occurs. These results validate proper functioning of the assay. In contrast, in conditions with a sufficient amount of certolizumab (1.42 μg and 142 ng, but not 14.2 ng) the color change is inhibited as evidenced by reduced absorbance compared to the positive control. Specifically, the bacterially-produced certolizumab reduced the signal to less than 0.1 at a concentration of 1.42 μg/mL and to a concentration of about 0.5 at 142 ng/ml.

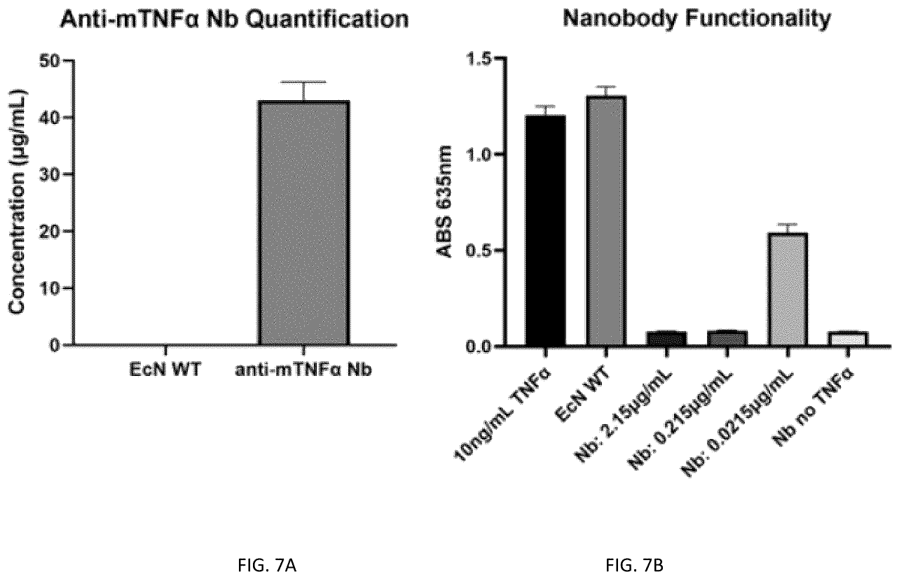

Effective bacterial production of the therapeutic agent ozoralizumab (anti-TNF-alpha nanobody) was also verified. Production is shown in FIG. 7A, and functionality is shown in FIG. 7B (tested using the HEK-blue functional assay visualized in the schematic of FIG. 6A). Engineered bacterial produced 43 μg/mL of nanobody from 1 liter of culture (FIG. 7A) and it was verified that the nanobody produced can bind to mTNFα and prevent color change in the HEK-blue assay when present in sufficient concentrations (FIG. 7B).

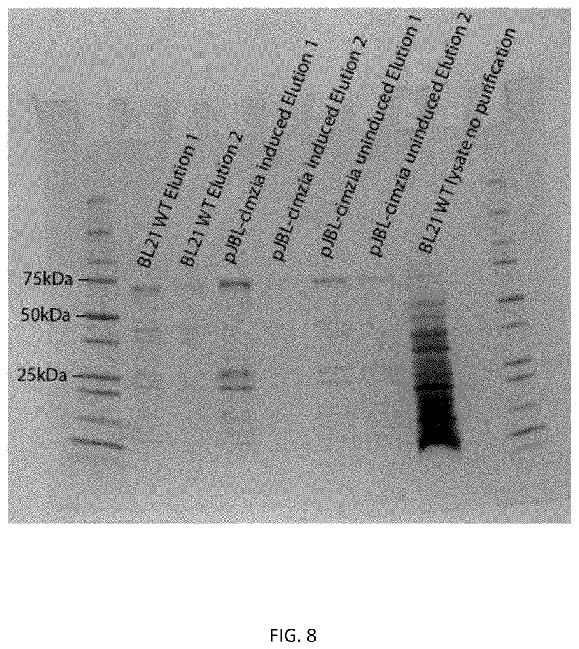

Production of the therapeutic agents was also achieved using the E. coli strain BL21. FIG. 8 shows successful production of certolizumab pegol (Cimzia®) in engineered BL21 bacteria, validated by western blot. Data shows purification of certrolizumab from BL21 WT and engineered calprotectin-responsive bacteria described herein (pJBL5034-lac-cimzia) induced and uninduced. The first two elution fractions of each were evaluated and the concentration equalized to 200 micrograms per milliliter using 12/8 BCA. The expected size of certolizumab pegol is 25 kDa.

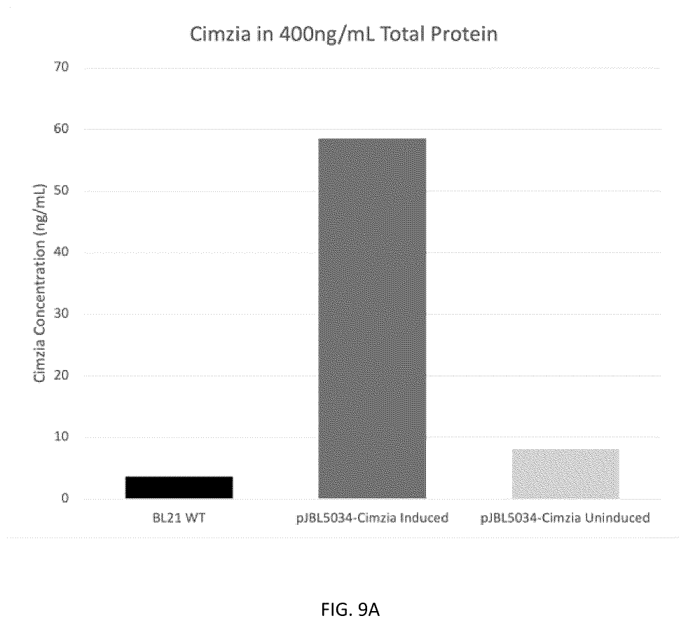

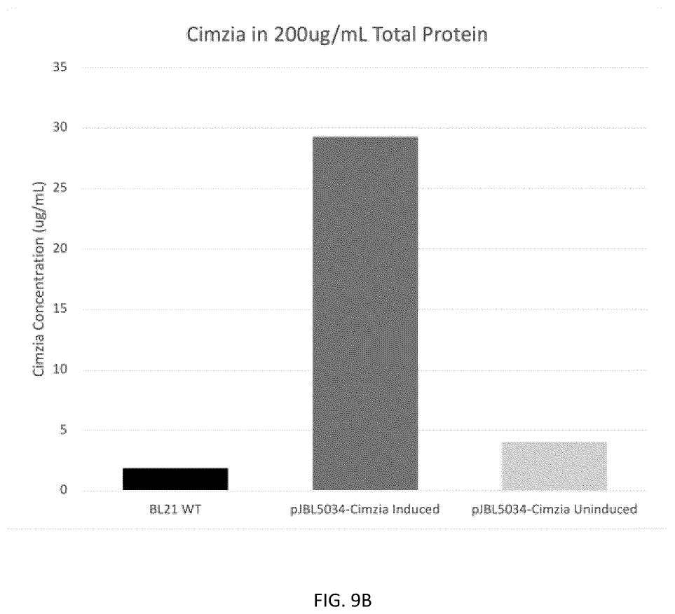

FIGS. 9A-9B show successful production of certolizumab pegol in engineered bacteria, validated by ELISA. Data shows purification of BL21 WT, an engineered calprotectin-responsive bacteria described herein (pJBL5034-lac-cimzia) induced and uninduced. The elution of each were evaluated and the concentration equalized to 200 micrograms per milliliter using 12/8 BCA and then diluted 500× with kit dilution buffer. FIG. 9A shows raw numbers from the ELISA and FIG. 9B shows data multiplied by the dilution factor.

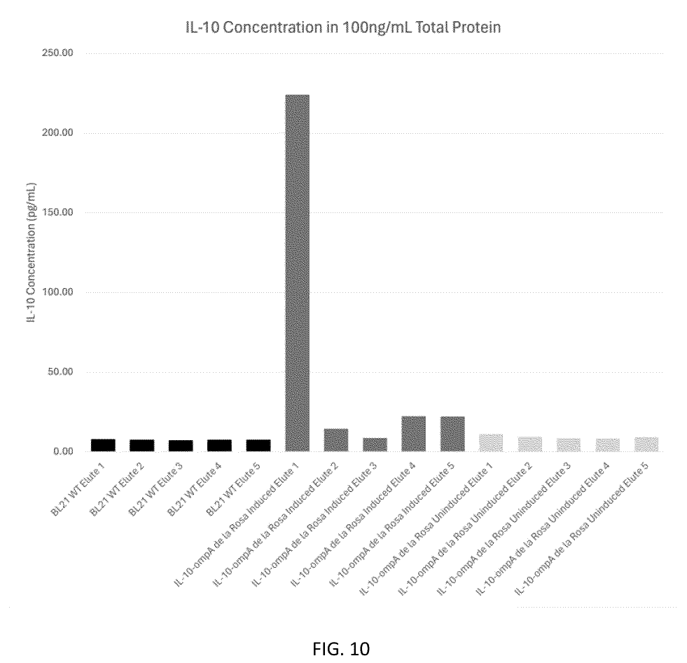

Bacteria were engineered herein to produce and selectively release the anti-inflammatory cytokine IL-10. E. coli cells were engineered to express IL-10. FIG. 10 shows successful production of IL-10 (e.g. IL-10 release) in engineered bacteria (pSC02-IL-10-ompA de la Rosa) expressing a tac promoter that drives expression of IL-10 and the bacteriophage-derived lysis element (174E) operatively linked to the calprotectin-responsive ykgMO promoter. IL-10 was conjugated to the ompA signaling peptide and a histidine tag for purification. Production was evaluated by ELISA. Data shows purification from BL21 WT and pSC02-IL-10-ompA de la Rosa in induced (calprotectin) and uninduced (no calprotectin) conditions. Dilution series of total concentrations of 100 ng/mL, 10 ng/mL, and 1 ng/mL were ran. Data demonstrates aproduction of IL-1 at concentrations of around 225 pg/mL of culture obtained from the first elute in the induced condition. As such, effective bacterial production and release of IL-10 in calprotectin-sufficient conditions was achieved.

To test the safety and efficacy of the bacterial therapeutics herein, animal models of IBD can be used. For example, a mammal having one or more symptoms of IBD can be orally dosed with the bacterial therapeutic and assessed for one or more symptoms of IBD, including body weight and damage to the gut epithelium.

Provided herein is an efficient, reliable, and non-invasive method of selectively releasing therapeutic proteins in response to inflammatory flares in the GI tract. These living medicines will improve patient outcomes, decrease healthcare costs, and expand care into patient populations lacking medical expertise in endoscopic evaluation of IBD. This therapeutic utilizes a calprotectin sensitive promoter to drive selective release of therapeutics in response to active inflammation. Certolizumab is an FDA approved monoclonal antibody directed against tumor-necrosis factor alpha for the treatment of IBD that is commercially produced by E. coli. Herein, certolizumab and the related nanobody ozoralizumab is selectively released during active inflammation, enabling earlier treatment of disease flares and maintaining remission.

1. An engineered bacterial therapeutic comprising a bacterial host cell expressing:
a) a protein for treatment of gastrointestinal disease, and
b) a lysis element operably linked to a calprotectin-responsive promoter,
wherein the lysis element induces lysis of the bacterial host cell and release of the protein for treatment of gastrointestinal disease in the presence of calprotectin.

2. The engineered bacterial therapeutic of claim 1, wherein the protein for treatment of gastrointestinal disease is an anti-inflammatory cytokine or an antibody.

3. The engineered bacterial therapeutic of claim 2, wherein the anti-inflammatory cytokine is interleukin-4 (IL-4), interleukin-5 (IL-5), interleukin-6 (IL-6), interleukin-10 (IL-10), interleukin-11 (IL-11), interleukin-13 (IL-13), interleukin-35 (IL-35), or interleukin-37 (IL-37).

4. The engineered bacterial therapeutic of claim 3, wherein the anti-inflammatory cytokine comprises a sequence having at least 90% identity to SEQ ID NO: 10.

5. The engineered bacterial therapeutic of claim 2, wherein the antibody is an anti-TNF-α antibody, an anti-IL-12 antibody, or an anti-IL-23 antibody.

6. The engineered bacterial therapeutic of claim 5, wherein the antibody comprises:
a) anti-TNF-α TNF antibody infliximab, adalimumab, golimumab, certolizumab, or ozoralizumab;
b) the anti-IL-12 antibody ustekinumab; or
c) the anti-IL-23 antibody guselkumab, brazikumab, mirikizumab, risankizumab, or tildrakizumab.

7. The engineered bacterial therapeutic of claim 1, wherein the lysis element is a phage-derived lysis element.

8. The engineered bacterial therapeutic of claim 7, wherein the phase-derived lysis element comprises an amino acid sequence having at least 90% identity with SEQ ID NO: 1.

9. The engineered bacterial therapeutic of claim 1, wherein the bacterial host cell comprises a probiotic bacterial strain.

10. The engineered bacterial therapeutic of claim 1, wherein the bacterial host cell comprises E. coli.

11. The engineered bacterial biosensor of claim 10, wherein the bacterial host cell comprises E. coli Nissle 1917 (EcN).

12. The engineered bacterial biosensor of claim 1, wherein the calprotectin-responsive promoter comprises ykgMO.

13. The engineered bacterial biosensor of claim 1, wherein the antibody for the treatment of gastrointestinal disease is conjugated to a signal peptide.

14. The engineered bacterial biosensor of claim 1, wherein the gastrointestinal disease comprises inflammatory bowel disease, a gastrointestinal infection, or Celiac disease.

15. A pharmaceutical composition comprising the engineered bacterial therapeutic of claim 1.

16. A method of treating gastrointestinal disease in a subject, comprising providing to the subject the pharmaceutical composition of claim 15.

17. The method of claim 16, wherein the gastrointestinal disease comprises inflammatory bowel disease, a gastrointestinal infection, or Celiac disease.

18. The method of claim 16, wherein the subject is a mammal.

19. The method of claim 16, wherein the subject is a human.
